(19) United States
(12) Patent Application Publication
(10) Pub. No.: US 2024/0032872 A1
(43) Pub. Date: Feb. 1, 2024

(54) BIOLOGICAL INFORMATION DETECTION DEVICE AND BIOLOGICAL INFORMATION DETECTION METHOD

(71) Applicant: Mitsubishi Electric Corporation, Tokyo (JP)

(72) Inventor: Yusuke NAKAMATSU, Tokyo (JP)

(73) Assignee: Mitsubishi Electric Corporation, Tokyo (JP)

(21) Appl. No.: 18/265,363

(22) PCT Filed: Feb. 5, 2021
(86) PCT No.: PCT/JP2021/004197

§ 371 Date: Jun. 5, 2023

(51) Int. Cl. A61B 5/00; A61B 5/05

(52) U.S. Cl. CPC A61B 5/7203; A61B 5/05; A61B 5/43

(57) ABSTRACT

Noise included in a signal related to biological information acquired by a radio wave sensor is reduced. A biological information detection device includes: a reflection signal acquiring unit to acquire a reflection signal, from a living body, of a radio wave emitted toward the living body; an image acquiring unit that acquires an image of the living body; an image processing unit that performs image processing on the acquired image; a signal strength estimating unit that estimates a possible range of signal strength of the reflection signal on the basis of a result of the image processing; and a noise reduction processing unit that reduces, from the reflection signal, a signal having strength outside the estimated range of signal strength.

### TECHNICAL FIELD

The present disclosure relates to a biological information detection device and a biological information detection method.

### BACKGROUND ART

In order to observe a health condition of a living body, research and development for acquiring biological information such as a respiration rate or a heart rate using a radio wave sensor has been performed. For example, Patent Literature 1 discloses a technique of acquiring a motion of a monitoring target as a motion signal using a radio wave sensor, converting the acquired motion signal into a frequency domain signal by fast Fourier transform, causing the frequency domain signal to pass through a band pass filter corresponding to a respiration rate or a heart rate, and obtaining a peak frequency as biological information such as a respiration rate or a heart rate.

### CITATION LIST

### Patent Literature

Patent Literature 1: JP 3057438 B2

### SUMMARY OF INVENTION

### Technical Problem

According to the technique disclosed in Patent Literature 1, when noise such as a body motion of a living body is input to a passband of a bandpass filter, a respiration rate or a heart rate is detected using a signal on which the noise is superimposed, and therefore there is a problem that erroneous detection occurs.

The present disclosure has been made in order to solve the above problem, and an object of one aspect of the embodiments is to provide a biological information detection device capable of reducing noise included in a signal related to biological information.

### Solution to Problem

A biological information detection device according to the present disclosure includes: a reflection signal acquiring unit to acquire a reflection signal, from a living body, of a radio wave emitted toward the living body; an image acquiring unit to acquire an image of the living body; an image processing unit to perform image processing on the acquired image; a signal strength estimating unit to estimate a possible range of signal strength of the reflection signal on a basis of a result of the image processing; and a noise reduction processing unit to reduce, from the reflection signal, a signal having strength outside the estimated range of signal strength.

### Advantageous Effects of Invention

According to the present disclosure, noise included in a signal related to biological information can be reduced.

### BRIEF DESCRIPTION OF DRAWINGS

FIG. 1 is a diagram illustrating a configuration example of a monitoring system including a biological information detection device.

FIG. 2 is a schematic diagram illustrating noise reduction processing.

FIG. 3A is a diagram illustrating a configuration example of hardware of the biological information detection device.

FIG. 3B is a diagram illustrating a configuration example of hardware of the biological information detection device.

FIG. 4 is a flowchart of a biological information detection method.

### DESCRIPTION OF EMBODIMENTS

### Embodiment 1

<Configuration>

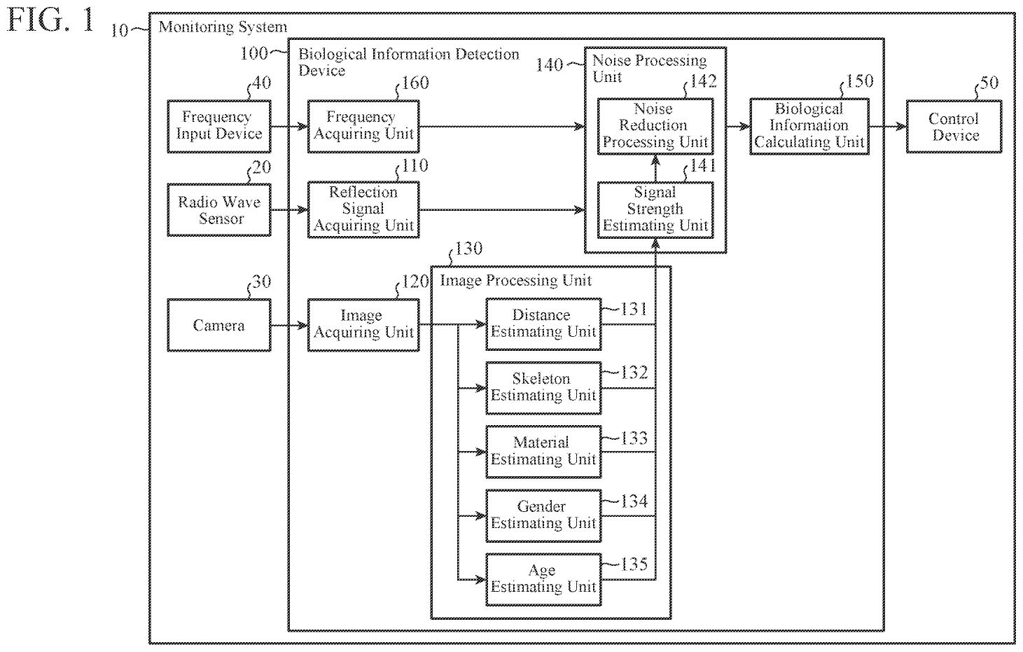

Hereinafter, various embodiments according to the present disclosure will be described in detail with reference to the drawings. FIG. 1 is a diagram illustrating a configuration example of a monitoring system 10 including a biological information detection device 100 according to Embodiment 1. The monitoring system 10 is a system for monitoring biological information such as a respiration rate or a heart rate. As illustrated in FIG. 1, the monitoring system 10 includes a radio wave sensor 20, a camera 30, a frequency input device 40, the biological information detection device 100, and a control device 50.

(Radio Wave Sensor)

The radio wave sensor 20 is a sensor for obtaining biological information from a monitoring target. The radio wave sensor 20 emits a radio wave toward a target, receives a reflected wave reflected by the target, amplifies a received reflection signal, and A/D-converts the amplified reflection signal. Examples of the radio wave sensor 20 include a Doppler sensor and a millimeter wave sensor. The radio wave sensor 20 may be disposed anywhere as long as the radio wave sensor 20 can emit a radio wave to a target and receive a reflection signal from the target. The radio wave sensor 20 outputs the A/D-converted reflection signal to the biological information detection device 100.

(Camera)

The camera 30 is a camera for acquiring an image of a target to be monitored. Examples of the camera 30 include a visible light camera and an infrared camera using a near-infrared light source and an infrared transmission filter. The camera 30 may be a monocular camera or a stereo camera. The camera 30 outputs image data obtained by imaging to the biological information detection device 100.

(Frequency Input Device)

The frequency input device 40 is an input device for designating a frequency domain in which frequency analysis is performed. The frequency input device 40 outputs the designated frequency domain to the biological information detection device 100.

(Biological Information Detection Device)

The biological information detection device 100 is a device that accepts signals from the radio wave sensor 20 and the camera 30, performs predetermined signal processing, and outputs a biological information signal. As illustrated in FIG. 1, the biological information detection device 100 includes a reflection signal acquiring unit 110, an image acquiring unit 120, a frequency acquiring unit 160, an image processing unit 130, a noise processing unit 140, and a biological information calculating unit 150.

The image processing unit 130 includes a distance estimating unit 131, a skeleton estimating unit 132, a material estimating unit 133, a gender estimating unit 134, and an age estimating unit 135.

The noise processing unit 140 includes a signal strength estimating unit 141 and a noise reduction processing unit 142.

(Reflection Signal Acquiring Unit)

The reflection signal acquiring unit 110 acquires the A/D-converted reflection signal from the radio wave sensor 20. As a result, a value for measuring biological information is detected. Specifically, for example, a value by a Doppler radar is detected. That is, a value corresponding to each frame is detected. Each frame corresponds to a predetermined time interval.

(Image Acquiring Unit)

The image acquiring unit 120 acquires the digital image data obtained by imaging by the camera 30. The image acquiring unit 120 outputs the acquired digital image data to the image processing unit 130.

(Frequency Acquiring Unit)

The frequency acquiring unit 160 acquires the designated frequency domain output by the frequency input device 40, and outputs the acquired designated frequency domain to the noise processing unit 140.

(Distance Estimating Unit)

The distance estimating unit 131 estimates a distance from the camera 30 to the target and the position of the target on the basis of the image acquired by the image acquiring unit 120. For the estimation of the distance and the position, a general algorithm such as trigonometry using a parallax by stereo vision can be used.

In a case of using a monocular camera, a size (for example, the number of pixels) of an object present in an imaging space, corresponding to a distance from the monocular camera to the object is defined in advance as table data. Then, the distance associated with the size of the object in the image obtained by imaging the object is acquired by referring to the table. This makes it possible to estimate the distance from the monocular camera to the target. In addition, by defining the position of a pixel of the object, corresponding to a distance from the monocular camera to the object, the position of the object can be estimated from the image obtained by imaging the object. Examples of the object present in the imaging space include a seat belt in a car interior space in which the camera 30 is disposed.

The distance estimating unit 131 estimates a distance from the radio wave sensor 20 to the target from the estimated position of the target and a positional relationship between the camera 30 and the radio wave sensor 20.

(Skeleton Estimating Unit)

The skeleton estimating unit 132 captures a posture or a skeleton of the target on the basis of the image acquired by the image acquiring unit 120, and acquires an angle and an area of a radio wave irradiation surface. As a method for estimating the posture or the skeleton, a general algorithm such as OpenPose can be used.

(Material Estimating Unit)

The material estimating unit 133 acquires a material of a radio wave irradiation surface of a wearing object such as clothes or accessories worn by the target on the basis of the image acquired by the image acquiring unit 120. The material can be estimated by creating a learning model in which an image and a material are associated with each other using a general algorithm such as convolutional neural network (CNN). By estimating the material, it is possible to consider a transmittance of a material present until the radio wave reaches a body surface of the target.

(Gender Estimating Unit)

The gender estimating unit 134 estimates a gender of the target on the basis of the image acquired by the image acquiring unit 120. The gender can be estimated by creating a learning model in which an image and a gender are associated with each other using a general algorithm such as convolutional neural network (CNN). By estimating the gender, it is possible to consider a reflectance of a radio wave on a body surface of the target due to a difference in gender.

(Age Estimating Unit)

The age estimating unit 135 estimates an age of the target on the basis of the image acquired by the image acquiring unit 120. The age can be estimated by creating a learning model in which an image and an age are associated with each other using a general algorithm such as convolutional neural network (CNN). By estimating the age, it is possible to consider a reflectance of a radio wave on a body surface of the target due to a difference in age.

(Signal Strength Estimating Unit)

The signal strength estimating unit 141 estimates a normally possible range of signal strength of a radio wave reflected by the target on the basis of estimation results output from the functional units included in the image processing unit 130. As an example, the signal strength estimating unit 141 estimates a range of signal strength of a radio wave reflected by the target in a normal state on the basis of the distance from the radio wave sensor 20 to the target estimated by the distance estimating unit 131. In general, it is conceivable that the shorter the distance is, the stronger the signal strength is. The term “normal” or “normal state” refers to a state in which the target does not intentionally move his or her body. Therefore, in a case where there is a natural body motion such as a pulse or a heartbeat, the target does not intentionally move his or her body, and therefore this case is included in the state.

As another example, the signal strength estimating unit 141 estimates a range of signal strength of a radio wave reflected by the target in a normal state on the basis of the estimation result of the posture or skeleton of the target estimated by the skeleton estimating unit 132. More specifically, the signal strength estimating unit 141 estimates the range of signal strength on the basis of the angle and area of the radio wave irradiation surface estimated by the skeleton estimating unit 132. In general, it is conceivable that the larger the degree of orthogonal intersection of the radio wave irradiation surface with respect to a boresight direction of an antenna of the radio wave sensor is, the stronger the signal strength is. In addition, it is conceivable that the larger the area of the irradiation surface viewed from the radio wave sensor is, the stronger the signal strength is.

As another example, the signal strength estimating unit 141 estimates a range of signal strength of a radio wave reflected by the target in a normal state on the basis of the material estimated by the material estimating unit 133. Since the transmittance of the radio wave varies depending on a material, it is conceivable that the higher the transmittance of a material of an object worn by the target is, the higher the signal strength is.

As another example, the signal strength estimating unit 141 estimates a range of signal strength of a radio wave reflected by the target in a normal state on the basis of the gender estimated by the gender estimating unit 134. On average, men have a higher water content than women. In addition, the higher the water content is, the higher the reflectance of a radio wave is. Therefore, since the reflectance of a radio wave is higher on average in men than in women, it is conceivable that the signal strength is higher in men than in women.

As another example, the signal strength estimating unit 141 estimates a range of signal strength of a radio wave reflected by the target in a normal state on the basis of the age estimated by the age estimating unit 135. In general, the lower the age is, the higher the water content is, and therefore it is conceivable that the lower the age is, the stronger the signal strength is.

These examples may be combined in any combination. For example, the signal strength estimating unit 141 may estimate a range of signal strength of a radio wave reflected by the target in a normal state on the basis of the estimated distance estimated by the distance estimating unit 131 and the angle and area of the irradiation surface estimated by the skeleton estimating unit 132. Alternatively, the signal strength estimating unit 141 may estimate a range of signal strength of a radio wave reflected by the target in a normal state on the basis of the estimated distance estimated by the distance estimating unit 131, the material estimated by the material estimating unit 133, the gender estimated by the gender estimating unit 134, or the age estimated by the age estimating unit 135

As a method for estimating a range of signal strength in a normal state, a method can be used in which a learning model in which the estimation results of the functional units included in the image processing unit 130 and the signal strength of a reflected wave coming from the target are associated with each other is created in advance using a general algorithm such as a convolutional neural network (CNN) under a transmission wave having a predetermined strength, and the range of signal strength in a normal state is estimated by inputting the estimation results of the functional units of the image processing unit 130 to the created learning model. A learning model using transmission waves having different levels of strength as inputs may be created. In addition, the range of signal strength of a reflected wave coming from the target in a normal state may be estimated with rule-based methodology using the estimation results of the functional units of the image processing unit 130 and using a general algorithm such as a 3σ method.

(Noise Reduction Processing Unit)

The noise reduction processing unit 142 reduces noise included in the reflection signal obtained by the reflection signal acquiring unit 110 using the range of signal strength of the target estimated by the signal strength estimating unit 141. As a noise reducing method, the reflection signal is subjected to fast Fourier transform, and a signal having strength exceeding the range of signal strength of the target estimated by the signal strength estimating unit 141 or a signal having strength less than the range is reduced or removed.

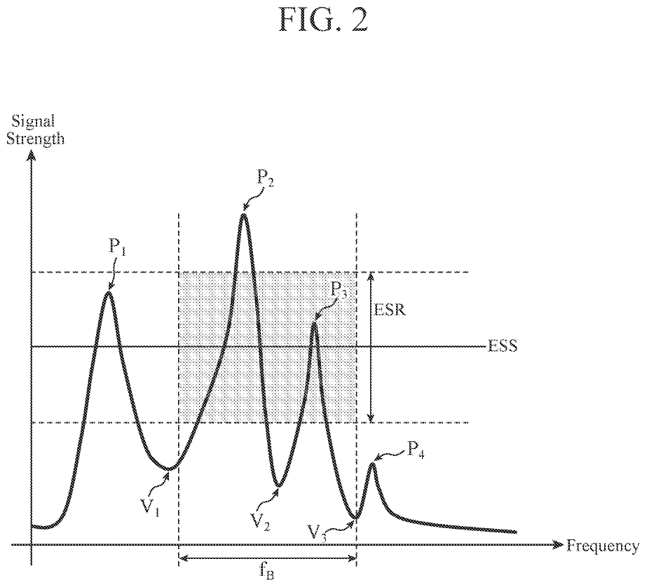

Here, signal strength estimation processing and noise reduction processing will be described with reference to FIG. 2. In FIG. 2, a waveform indicates a frequency spectrum of the signal obtained from the target. The signal strength estimating unit 141 estimates a predetermined range from estimated signal strength ESS of the target as an estimated strength range ESR on the basis of the estimation results of the functional units of the image processing unit 130. The noise reduction processing unit 142 reduces or removes a signal having strength exceeding an upper limit value of the estimated strength range ESR or a signal having strength less than a lower limit value of the estimated strength range ESR in a designated frequency domain f_{B}, regarding the signal as unintentional one.

The frequency domain f_{B} is designated by a user via the frequency input device 40. The designated frequency domain f_{B} is acquired by the frequency acquiring unit 160 from the frequency input device 40 and supplied to the noise processing unit 140. In a normal state, a possible range of frequencies varies depending on a parameter related to biological information, such as a heart rate of 0.8 to 1.2 [Hz] and a respiration rate of 0.2 to 0.3 [Hz]. Therefore, the frequency domain f_{B} is designated depending on information desired to be acquired.

In the waveform of FIG. 2, since a second peak P₂ from the left exceeds the estimated strength range ESR, it is conceivable that noise due to, for example, body motion is superimposed on the peak Pa. Therefore, the noise reduction processing unit 142 replaces a value of signal strength from a valley V₁ to a valley V₂ with, for example, 0. Alternatively, the value of the signal strength from the valley V₁ to the valley V₂ is multiplied by an appropriate coefficient. In this way, noise is removed or reduced.

After performing the noise reduction processing as described above, the noise reduction processing unit 142 outputs a reflection signal from which noise has been reduced or removed to the biological information calculating unit 150.

(Biological Information Calculating Unit)

The biological information calculating unit 150 calculates biological information from the reflection signal that has been subjected to the noise reduction processing by the noise reduction processing unit 142. As for a calculation method, the calculation can be performed by specifying a peak frequency in a possible frequency band of any piece of biological information from the reflection signal that has been subjected to the noise reduction processing. As a specific example, the biological information calculating unit 150 specifies a frequency corresponding to a peak P₃ in the designated frequency domain f_{B}. Then, predetermined calculation is performed on the specified frequency to obtain biological information. For example, in a case where 0.8 to 1.2 [Hz] corresponding to a heart rate is designated as the frequency domain f_{B}, it is assumed that, for example, 1 [Hz] is specified as the frequency corresponding to the peak P₃. In this case, 1 is multiplied by 60, and a heart rate of 60 [times/min] is calculated as the biological information. Examples of the biological information to be calculated include a pulse rate, a respiration rate, and a heart rate variation in addition to the heart rate. The biological information calculating unit 150 outputs the calculated biological information to the control device 50.

Note that data that is not within the range of the frequency domain f_{B} is not considered. That is, in the waveform of FIG. 2, frequencies corresponding to a first peak P₁ from the left and a fourth peak P₄ from the left are not considered. In addition, since noise has been reduced or removed from the signal of the peak P₂, the frequency corresponding to the peak P₂ is not used.

The control device 50 performs various controls using the biological information received from the biological information calculating unit 150. For example, display control of displaying biological information on a display device (not illustrated) is performed.

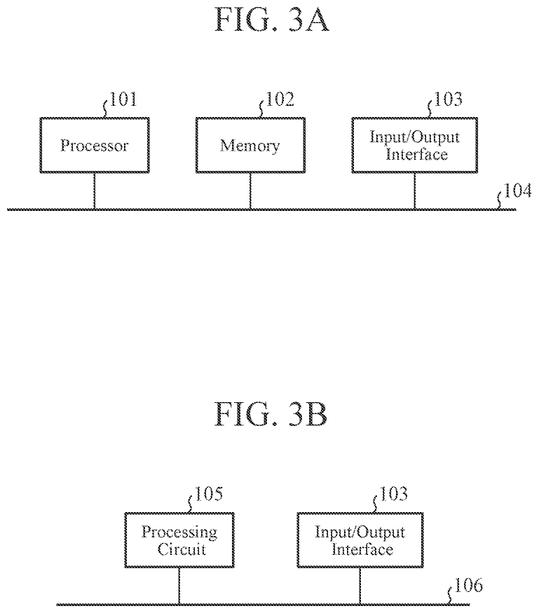

Next, configuration examples of hardware of the data biological information detection device 100 will be described with reference to FIGS. 3A and 3B. As an example, as illustrated in FIG. 3A, the biological information detection device 100 includes a processor 101, a memory 102, and an input/output interface 103. The processor 101, the memory 102, and the input/output interface 103 are connected to each other via a bus 104. In the case of this configuration example, the input/output interface 103 implements the reflection signal acquiring unit 110, the image acquiring unit 120, and the frequency acquiring unit 160. In addition, the functional units of the image processing unit 130, the functional units of the noise processing unit 140, and the biological information calculating unit 150 are implemented by a program stored in the memory 102 being read and executed by the processor 101. The program is implemented as software, firmware, or a combination of software and firmware. Examples of the memory 102 include a nonvolatile or volatile semiconductor memory such as a random access memory (RAM), a read only memory (ROM), a flash memory, an erasable programmable read only memory (EPROM), or an electrically-EPROM (EEPROM), a magnetic disk, a flexible disk, an optical disc, a compact disc, a mini disc, and a DVD.

As another example, as illustrated in FIG. 3B, the biological information detection device 100 includes a processing circuit 105 and the input/output interface 103. The processing circuit 105 and the input/output interface 103 are connected to each other via a bus 106. In the case of this configuration example, the input/output interface 103 implements the reflection signal acquiring unit 110, the image acquiring unit 120, and the frequency acquiring unit 160. In addition, the processing circuit 105 implements the functional units of the image processing unit 130, the functional units of the noise processing unit 140, and the biological information calculating unit 150. Examples of the processing circuit 105 include a single circuit, a composite circuit, a programmed processor, a parallel programmed processor, an application specific integrated circuit (ASIC), a field-programmable gate array (FPGA), and a combination thereof. The functions of the functional units of the image processing unit 130, the functional units of the noise processing unit 140, and the biological information calculating unit 150 may be implemented by separate processing circuits, or these functions may be collectively implemented by one processing circuit.

<Operation>

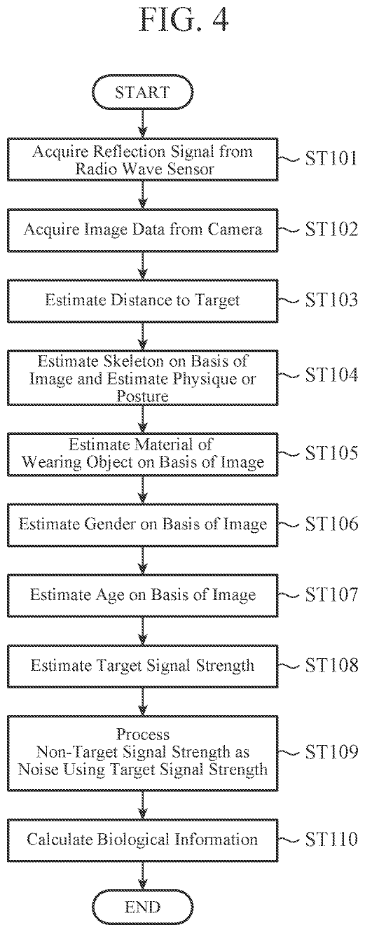

Next, a flow of a biological information detection method performed as an operation by the biological information detection device 100 will be described with reference to FIG. 4. In step S101, the reflection signal acquiring unit 110 acquires, from the radio wave sensor 20, a reflection signal derived from a living body as a target, the reflection signal being acquired by the radio wave sensor 20. In step S102, the image acquiring unit 120 acquires, from the camera 30, image data of the target acquired by the camera 30. Note that the processing in step S101 and the processing in step S102 are in no particular order.

In step S103, the distance estimating unit 131 of the image processing unit 130 estimates a position of the target on the basis of the image data acquired by the image acquiring unit 120. In addition, the distance estimating unit 131 estimates a distance from the radio wave sensor 20 to the target from the estimated position of the target and a positional relationship between the camera 30 and the radio wave sensor 20. In step S104, the skeleton estimating unit 132 of the image processing unit 130 captures a posture or a skeleton of the target on the basis of the image acquired by the image acquiring unit 120, and acquires an angle and an area of a radio wave irradiation surface. In step S105, the material estimating unit 133 of the image processing unit 130 acquires a material of a wearing object such as clothes or accessories worn by the target on the radio wave irradiation surface on the basis of the image acquired by the image acquiring unit 120. In step S106, the gender estimating unit 134 of the image processing unit 130 estimates a gender of the target on the basis of the image acquired by the image acquiring unit 120. In step S107, the age estimating unit 135 of the image processing unit 130 estimates an age of the target on the basis of the image acquired by the image acquiring unit 120. Note that the processing from step S103 to step S107 is in no particular order.

In step S108, the signal strength estimating unit 141 estimates a possible range of signal strength of a radio wave reflected by the target on the basis of the estimation results estimated by the functional units of the image processing unit 130. In step S109, the noise reduction processing unit 142 processes signal strength derived from a non-target included in the reflection signal obtained by the reflection signal acquiring unit 110 as noise using the signal strength of the target obtained by the signal strength estimating unit 141.

In step S110, the biological information calculating unit 150 calculates biological information from the reflection signal that has been subjected to the noise reduction processing by the noise reduction processing unit 142.

According to the biological information detection device 100 of the present disclosure, in a case where a plurality of peaks is observed in a designated frequency band including biological information desired to be extracted when a reflection signal is subjected to fast Fourier transform, a signal having strength outside an estimated signal strength range of a target is removed or reduced, and therefore erroneous detection of biological information can be reduced. Therefore, detection accuracy of biological information can be improved.

<Supplementary Note>

Some of various aspects of the embodiments described above are summarized below.

(Supplementary Note 1)

A biological information detection device 100 according to supplementary note 1 includes: a reflection signal acquiring unit 110 that acquires reflection signal, from a living body, of a radio wave emitted toward the living body; an image acquiring unit 120 that acquires an image of the living body; an image processing unit 130 that performs image processing on the acquired image; a signal strength estimating unit 141 that estimates a possible range of signal strength of the reflection signal on the basis of a result of the image processing; and a noise reduction processing unit 142 that reduces, from the reflection signal, a signal having strength outside the estimated range of signal strength.

(Supplementary Note 2)

A biological information detection device 100 according to supplementary note 2 is the biological information detection device 100 according to supplementary note 1, in which the image processing unit includes a skeleton estimating unit 132 that estimates a skeleton of the living body, and the signal strength estimating unit estimates the possible range of signal strength of the reflection signal on the basis of the estimated skeleton.

(Supplementary Note 3)

A biological information detection device 100 according to supplementary note 3 is the biological information detection device 100 according to supplementary note 1 or 2, in which the image processing unit includes a material estimating unit 133 that estimates a material of a wearing object of the living body, and the signal strength estimating unit estimates the possible range of signal strength of the reflection signal on the basis of the estimated material.

(Supplementary Note 4)

A biological information detection device 100 according to supplementary note 4 is the biological information detection device 100 according to any one of supplementary notes 1 to 3, in which the image processing unit includes a gender estimating unit 134 that estimates a gender of the living body, and the signal strength estimating unit estimates the possible range of signal strength of the reflection signal on the basis of the estimated gender.

(Supplementary Note 5)

A biological information detection device 100 according to supplementary note 5 is the biological information detection device 100 according to any one of supplementary notes 1 to 4, in which the image processing unit includes an age estimating unit 135 that estimates an age of the living body, and the signal strength estimating unit estimates the possible range of signal strength of the reflection signal on the basis of the estimated age.

(Supplementary Note 6)

A biological information detection device 100 according to supplementary note 6 is the biological information detection device 100 according to any one of supplementary notes 1 to 5, in which the image processing unit includes a distance estimating unit 131 that estimates a distance to the living body, and the signal strength estimating unit estimates the possible range of signal strength of the reflection signal on the basis of the estimated distance.

(Supplementary Note 7)

A biological information detection method according to supplementary note 7 is a biological information detection method performed by a biological information detection device including a reflection signal acquiring unit, an image acquiring unit, an image processing unit, a signal strength estimating unit, and a noise reduction processing unit, the biological information detection method including: step S101 in which the reflection signal acquiring unit acquires a reflection signal, from a living body, of a radio wave emitted toward the living body; step S102 in which the image acquiring unit acquires an image of the living body; steps (S103 to S107) in which the image processing unit performs image processing on the acquired image; step S108 in which the signal strength estimating unit estimates a possible range of signal strength of the reflection signal on the basis of a result of the image processing; and step S109 in which the noise reduction processing unit reduces, from the reflection signal, a signal having strength outside the estimated range of signal strength.

Note that the embodiments can be combined, and each of the embodiments can be appropriately modified or omitted.

### INDUSTRIAL APPLICABILITY

The biological information detection device according to the present disclosure can reduce noise superimposed on a signal of biological information, and therefore can be used in a system that monitors biological information.

### REFERENCE SIGNS LIST

10: monitoring system, 20: radio wave sensor, 30: camera, 40: frequency input device, 50: control device, 100: biological information detection device, 101: processor, 102: memory, 103: input/output interface, 104: bus, 105: processing circuit, 106: bus, 110: reflection signal acquiring unit, 120: image acquiring unit, 130: image processing unit, 131: distance estimating unit, 132: skeleton estimating unit, 133: material estimating unit, 134: gender estimating unit, 135: age estimating unit, 140: noise processing unit, 141: signal strength estimating unit, 142: noise reduction processing unit, 150: biological information calculating unit, 160: frequency acquiring unit

1. (canceled)

2. A biological information detection device comprising:
processing circuitry to acquire a reflection signal, from a living body, of a radio wave emitted toward the living body;
to acquire an image of the living body;
to perform image processing on the acquired image;
to estimate a possible range of signal strength of the reflection signal on a basis of a result of the image processing; and
to reduce, from the reflection signal, a signal having strength outside the estimated range of signal strength, wherein
the processing circuitry is further configured to estimate a skeleton of the living body, and
to estimate the possible range of signal strength of the reflection signal on a basis of the estimated skeleton.

3. A biological information detection device comprising:
processing circuitry
to acquire a reflection signal, from a living body, of a radio wave emitted toward the living body;
to acquire an image of the living body;
to perform image processing on the acquired image;
to estimate a possible range of signal strength of the reflection signal on a basis of a result of the image processing; and
to reduce, from the reflection signal, a signal having strength outside the estimated range of signal strength, wherein
the processing circuitry is further configured to estimate a material of a wearing object of the living body, and
to estimate the possible range of signal strength of the reflection signal on a basis of the estimated material.

4. A biological information detection device comprising:
processing circuitry
to acquire a reflection signal, from a living body, of a radio wave emitted toward the living body;
to acquire an image of the living body;
to perform image processing on the acquired image;
to estimate a possible range of signal strength of the reflection signal on a basis of a result of the image processing; and
to reduce, from the reflection signal, a signal having strength outside the estimated range of signal strength, wherein
the processing circuitry is further configured to estimate a gender of the living body, and
to estimate the possible range of signal strength of the reflection signal on a basis of the estimated gender.

5. A biological information detection device comprising:
processing circuitry
to acquire a reflection signal, from a living body, of a radio wave emitted toward the living body;
to acquire an image of the living body;
to perform image processing on the acquired image;
to estimate a possible range of signal strength of the reflection signal on a basis of a result of the image processing; and
to reduce, from the reflection signal, a signal having strength outside the estimated range of signal strength, wherein
the processing circuitry is further configured to estimate an age of the living body, and
to estimate the possible range of signal strength of the reflection signal on a basis of the estimated age.

6. A biological information detection device comprising:
processing circuitry
to acquire a reflection signal, from a living body, of a radio wave emitted toward the living body;
to acquire an image of the living body;
to perform image processing on the acquired image;
to estimate a possible range of signal strength of the reflection signal on a basis of a result of the image processing; and
to reduce, from the reflection signal, a signal having strength outside the estimated range of signal strength, wherein
the processing circuitry is further configured to estimate a distance to the living body, and
to estimate the possible range of signal strength of the reflection signal on a basis of the estimated distance.

7. A biological information detection method comprising:
acquiring a reflection signal, from a living body, of a radio wave emitted toward the living body;
acquiring an image of the living body;
performing image processing on the acquired image to estimate a skeleton, a material of a wearing object, a gender, or an age of the living body;
estimating a possible range of signal strength of the reflection signal on a basis of a result of the image processing; and
reducing from the reflection signal, a signal having strength outside the estimated range of signal strength.
